(19) United States
(12) Patent Application Publication
(10) Pub. No.: US 2011/0033940 A1
(43) Pub. Date: Feb. 10, 2011

(54) CLICK CHEMISTRY, MOLECULAR TRANSPORT JUNCTIONS, AND COLORIMETRIC DETECTION OF COPPER

(75) Inventors: Chad A. Mirkin, Wilmette, IL (US); Xiaodong Chen, Singapore (SG); Adam B. Braunschweig, Evanston, IL (US); Michael J. Wiester, Evanston, IL (US); Xiaoyang Xu, Evanston, IL (US); Weston L. Daniel, Evanston, IL (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: NORTHWESTERN UNIVERSITY, Evanston, IL (US)

(21) Appl. No.: 12/696,638

(22) Filed: Jan. 29, 2010

Related U.S. Application Data
(60) Provisional application No. 61/148,704, filed on Jan. 30, 2009.
(60) Provisional application No. 61/163,081, filed on Mar. 25, 2009.

(51) Int. Cl. G01N 33/50; H01B 1/12

(52) U.S. Cl. 436/80; 252/500

(57) ABSTRACT

Click chemistry is used to construct molecular transport junctions (MTJs) through assembly of a molecular wire across a nanogap formed between two electrodes. Also disclosed are methods of using click chemistry and oligonucleotide-modified nanoparticles to detect the presence of copper in a sample.

### CROSS REFERENCE TO RELATED APPLICATIONS

This application claims the benefit of U.S. Provisional Application No. 61/148,704, filed Jan. 30, 2009 and U.S. Provisional Application No. 61/163,081, filed Mar. 25, 2009, the disclosure of each incorporated herein by reference in its entirety.

### STATEMENT OF GOVERNMENTAL SUPPORT

This invention was made with U.S. government support under the National Science Foundation (NSEC) Grant No. EEC-0647560, Fleet and Industrial Supply Center San Diego NSSEFF Award N00244-09-1-0012, and U.S. Army Medical Research and Material Command Grant No. W81XWH-08-1-0766. The government has certain rights in this invention.

### BACKGROUND

Click chemistry is a facile way in which to prepare compounds, including oligomeric and polymeric compounds, using modular starting materials. Click chemistry refers to a reaction between an alkyne and an azide to form an azole, as shown in below in Scheme 1.

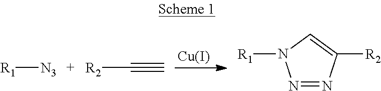

### SUMMARY

Disclosed herein are compositions comprising a nanowire and a compound capable of spanning a gap in the nanowire. More specifically, disclosed herein are composition comprising (a) a nanowire comprising two segments separated by a gap of about 2 nm to about 20 nm and a coating disposed along one side of the nanowire, and (b) a compound of formula (I) having a length sufficient to span the gap and having a structure I:

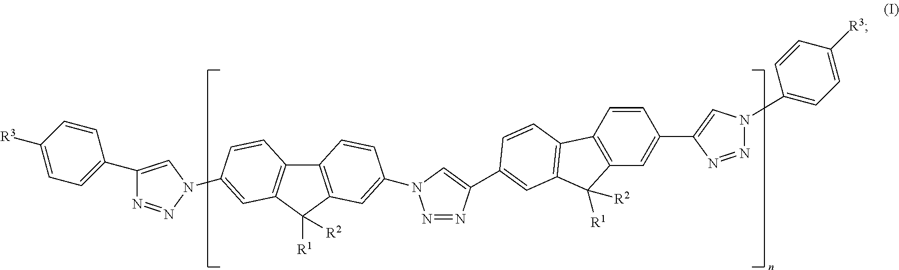

wherein R¹ and R² are independently selected from the group consisting of hydrogen, C₁-C₂₀alkyl, and C₁-C₂₀alkylaryl, each R³ is a moiety or residue of a moiety capable of forming a covalent bond or non-covalent interaction with a segment of the nanowire; and n is an integer of 1 to 20. In various cases, the compound of formula (I) spans the gap of the nanowire. In various cases, an electrode is connected to each segment, or at least two segments, of the nanowire. In some cases, the nanowire comprises a third segment and a second gap.

R¹ and R² can be the same or different. In some cases, at least one of R¹ and R² is C₁-C₂₀alkyl. In some cases, the gap is about 2 nm to 10 nm. In various embodiments, at least one segment comprises a metal, such as gold, platinum, palladium, copper, silver, nickel, titanium, or a mixture thereof. In some embodiments, at least one segment comprises a polymer, such as polypyrrole, polyaniline, polythiophene, poly(ethylenedioxy)thiophene, poly(heteraromic vinylene), polyvinylphosphate, or a mixture thereof. In a specific embodiment, at least one R³ comprises sulfur and at least one segment comprises gold. The coating can be silica.

In another aspect, disclosed herein is a method of making a composition as described above. The method comprises admixing the nanowire, a compound of formula (II), a compound of formula (III), a compound of formula (IV), and a copper (I) salt to form the compound of formula (I), wherein the compound of formula (I) spans the gap of the nanowire

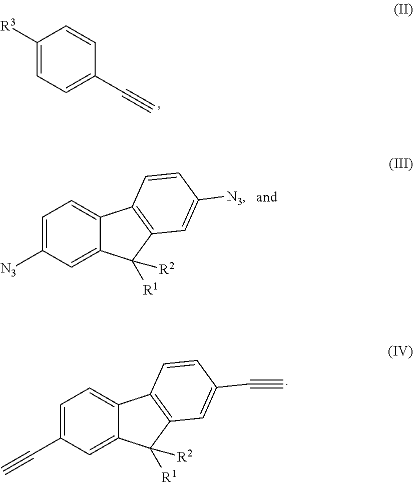

In some cases, the copper (I) salt is generated in situ from a copper (II) salt and a reducing agent. The reducing agent can be ascorbic acid or a salt thereof.

In some embodiments, the admixing comprises a sequence of:

(i) the compound of formula (II), the copper (I) salt, and nanowire are admixed to form a first intermediate,

(ii) the first intermediate is admixed with the compound of formula (III) in the presence of the copper (I) salt to form a second intermediate,

(iii) the second intermediate is admixed with the compound of formula (IV) in the presence of the copper (I) salt,

wherein steps (ii) and (iii) are repeated until the compound of formula (I) is formed to span the gap of the nanowire. In various cases, the method further comprises detecting the formation of the composition by monitoring an amount of current that passes through the nanowire.

In another aspect, provided herein is a method of conducting current comprising passing current through a nanowire composition as disclosed herein.

In yet another aspect, provided herein is a method of detecting copper in a sample, comprising (a) heating the sample and a complex to determine a melting temperature of the complex in the presence of the sample, the complex comprising (i) a first oligonucleotide attached to a first nanoparticle, (ii) a second oligonucleotide attached to a second nanoparticle, and (iii) a third oligonucleotide; and (b) comparing the melting temperature of the complex in the presence of the sample to a melting temperature of the complex in the absence of copper, wherein when the complex has a higher melting temperature in the presence of the sample than in the absence of copper, the sample comprises copper; and wherein each of the second and third oligonucleotide is sufficiently complementary to the first oligonucleotide to hybridize; the second oligonucleotide is complementary to a first portion of the first oligonucleotide; the third oligonucleotide is complementary to a second portion of the first oligonucleotide; one of the second oligonucleotide or third oligonucleotide comprises an alkyne moiety at a terminus and the other of the second oligonucleotide or third oligonucleotide comprises an azide moiety at a terminus; the alkyne moiety and the azide moiety react in the presence of copper to ligate the second oligonucleotide and third oligonucleotide; and the first portion of the first oligonucleotide is sufficiently adjacent to the second portion of the first oligonucleotide to permit ligation between the first oligonucleotide and the second oligonucleotide.

The complex can be heated in the presence of a copper ligand, a reducing agent or both. In some cases, the copper ligand comprises tris-triazolylamine, bathophenanthroline disulfonic acid, N,N,N′,N′,N″-pentamethyldiethylenetriamine, N,N-dimethylcyclohexane-1,2-diamine, N,N,N-trimethylethane-1,2-diamine, a bipyridine, 1,10-phenanthroline, or mixtures thereof. In various cases, the reducing agent comprises ascorbic acid, an ascorbate salt, sodium borohydride, 2-mercaptoethanol, dithiothreitol (DTT), hydrazine, lithium aluminum hydride, diisobutylaluminum hydride, oxalic acid, Lindlar catalyst, a sulfite compound, a stannous compound, a ferrous compound, sodium amalgam, tris(2-carboxyethyl)phosphine, hydroquinone, and mixtures thereof. In some cases, the methods disclosed herein can be used to detect copper at a concentration of at least 20 μM, or 20 μM to 100 μM. In various cases the sample comprises copper (II) or copper (I). In a specific case, the sample comprises copper (I) and copper (II).

In various embodiments, at least one of the first nanoparticle or second nanoparticle comprises a metal, such as gold. In some cases, at least one or each of the first nanoparticle or second nanoparticle has a diameter of 10 nm to 200 nm.

In some embodiments, the melting temperature of the complex in the presence of the sample is at least 3° C. or at least 5° C. higher than the melting temperature in the absence of copper.

In still another aspect, provided herein are methods of detecting copper in a sample comprising (a) heating the sample and a complex comprising (1) a first oligonucleotide attached to a first nanoparticle, (2) a second oligonucleotide attached to a second nanoparticle, and (3) a third oligonucleotide; and (b) heating the complex in the absence of copper, wherein when the complex in the absence of copper has a change in color or absorbance before the complex in the presence of the sample has a change in color or absorbance, the sample comprises copper; and wherein each of the second and third oligonucleotide is sufficiently complementary to the first oligonucleotide to hybridize; the second oligonucleotide is complementary to a first portion of the first oligonucleotide; the third oligonucleotide is complementary to a second portion of the first oligonucleotide; one of the second oligonucleotide or third oligonucleotide comprises an alkyne moiety at a terminus and the other of the second oligonucleotide or third oligonucleotide comprises an azide moiety at a terminus; the alkyne moiety and the azide moiety react in the presence of copper to ligate the second oligonucleotide and third oligonucleotide; and the first portion of the first oligonucleotide is sufficiently adjacent to the second portion of the first oligonucleotide to permit ligation between the first oligonucleotide and the second oligonucleotide.

In various cases, the change in color is from colorless or light purple to red. The color change can be monitored by measuring an absorbance of the complex as the complex is heated.

In some cases for the methods disclosed herein, the change in absorbance or change in melting temperature indicates the copper concentration in the sample.

### BRIEF DESCRIPTION OF THE FIGURES

FIG. 1 shows (A) a schematic illustration of click chemistry within the nanogaps of a nanowire; (B) molecules used herein; and (C) a schematic of a nanowire used in the methods disclosed herein, depicting segments, gaps, and the coating of the nanowire.

FIG. 2 shows (A) a scanning electron microscopy (SEM) image of 2 nm OWL-generated nanogap; (B) an SEM image of 2 nm nanogap-molecular transport junction (MTJ) device; and (C) a representative current-voltage (I-V) response for 2 nm OWL-fabricated gaps before (solid square curve), after (open uptriangle curve) modified with a alkynyl benzene thiol (compound 1), and the bridging click reaction of the diazido compound 2 with compound 1 (open downtriangle curve), and a theoretical fitting I-V curve.

FIG. 3 shows (A) an SEM image of an OWL generated nanowire with a 100 nm nanogap; (B) 3D confocal scanning Raman images of an OWL generated gap structure modified with compound 1, where the optical image (inset of b), which is simultaneously obtained, shows the position of the nanogap (scale bar 2 μm); and (C) representative Raman spectrum taken for the different steps: (i) compound 1 modified within gap; (ii) following addition of compound 2; and (iii) following addition of di-alkynyl compound 3.

FIG. 4 shows representative I-V response for the multistep click reactions within OWL-fabricated gaps before and after the last step click reaction, (A) 5 nm OWL-gap, two step click reaction; and (B) 7 nm OWL-fabricated gap, three step click reaction.

FIG. 5 shows (a) an alkyne-terminated compound bound to a gold surface via a thiol bond that reacts with a diazide compound 2 in the presence of a Cu(I) catalyst to form a 1,2,3-triazole linkage; the XPS characterization following the click chemistry on the gold substrate surface of the S band is shown in 5(b) and the N band in 5(c).

FIG. 6 shows a control experiment of a triazole product of click chemistry (compound 4) assembled in a 2 nm gap in a nanowire and the resulting I-V response.

FIG. 7 (top) shows the Raman spectrum of di-alkynyl compound 3; and (bottom) of compound 1.

FIG. 8 shows the Raman spectrum of click chemistry triazole compound 4.

FIG. 9 shows a schematic representation of the formation of a MTJ using click chemistry to bridge a gap of a nanowire, and the Raman spectrum of the nanowire.

FIG. 10 shows a schematic illustration representing the aggregation and dissociation pathway of the gold nanoparticle probes used in the colorimetric detection of copper ion.

FIG. 11 shows melting curves of the gold nanoparticle aggregates in the presence of Cu²⁺ at concentrations from 0 μM to 500 μM. The change in the extinction was monitored at 525 nm.

FIG. 12 shows melting curves of the gold nanoparticle aggregates in the presence of various metal ions. The change in extinction was monitored at 525 nm.

FIG. 13 shows the change in melting temperature (Tₘ ° C.) of hybridized oligonucleotide-nanoparticle aggregates in the presence of various copper concentrations.

### DETAILED DESCRIPTION

The development of efficient methods for constructing MTJs having a capability to spectroscopically identify molecules assembled within the junctions continues to challenge the field of molecular electronics.[1,2] Most of the current work in MTJ fabrication relies primarily on ex situ syntheses of molecular wires (e.g., dithiolated molecules) followed by subsequent insertion of the molecules into the gap devices.[3] The problems associated with this approach are: 1) the difficulty involved in synthesizing long molecular wires with a thiol on each end because of the low stability and synthetic yields of these molecules, and 2) complications in bridging the electrodes because of a strong tendency of such molecular wires to aggregate.[4] In addition, the small junction sizes (normally only several nanometers in width) often prohibit the use of routine spectroscopic tools to identify the contents within MTJs.

Therefore, a modular method for in situ synthesis of molecular wires to bridge nanogaps[4,5] that allows spectroscopic tracking of the assembly process merits development. A new method to fabricate MTJs using the alkyne-azide “click reaction” within nanogaps fabricated by On-Wire Lithography (OWL), using surface enhanced Raman scattering (SERS) to characterize the assembly processes within the gaps, is disclosed herein. This strategy for forming MTJs proceeds in high yields, and, as a result of the accessible functional group requirements of click chemistry, is a modular approach that can be used to form MTJs comprised of different molecular components. Additionally, this approach is well suited for studying transport properties of various molecular architectures because the resulting triazole formed by reacting the alkyne and azide groups retains the conjugation required for the electronic transport.

OWL is an electrochemistry-based nanofabrication technique used to prepare a wide variety of nanowire-based structures (e.g., nanogaps and disk arrays) with control over composition and morphology.[6] The obtained structures have been used for prototyping nanostructured materials with advanced functions in the context of molecular electronics[6-8] and SERS.[9-11] OWL allows one to prepare gaps with feature size control down to 2 nm, which makes them promising testbeds for fabricating MTJs.[7] The characteristics of OWL-fabricated nanogaps include high-throughput and tunable, molecular-sized features.

Click chemistry is used to demonstrate the in situ modular synthesis of molecular wires within the OWL-generated nanogaps. Click chemistry is a synthetic approach involving reactions that proceed quickly, with high yields and specificity, under mild conditions.[12] An advantage of forming molecular wires using the click methodology within the OWL-generated nanogaps is that in situ fabrication within a confined space (nanogap) is challenging for other existing testbeds, such as scanning probes [13-15], and wire crossing[16] because these techniques are not easily solution-processable. Mechanical break junction techniques [17,18], on the other hand, provide only limited control over gap size in comparison to nanogaps formed by OWL. In this study, the CuI-catalyzed 1,3-dipolar Hüisgen cycloaddition (click reaction) between azides and alkynes is utilized as a model reaction for preparing molecular wires within OWL-generated nanogaps to form MTJs.

Thus, disclosed herein are nanowires having metal segments and gaps, wherein a compound fills the gaps and has a length sufficient to span the gap length, e.g., within about 20% of the length of the gap. In some cases, the length of the compound is about 85% to about 115%, about 90% to about 115%, about 90% to about 110%, about 100% to about 120% that of the gap. The compound can be a polyunsaturated compound, and preferably has a conjugated π-system. The nanowires can be prepared using known on-wire lithography (OWL) techniques, as discussed in L. Qin et al., Science 309, 113 (2005) and U.S. Pat. No. 7,252,698, each of which is incorporated by reference in its entirety.

As used herein, the term “nanowire” refers to segments of electroactive materials separated by gaps and held together with a coating along one side. The electroactive materials are materials capable of conducting a current, such as metal or polymers having a charge (e.g., polypyrrole). The metal can be any metal, or combination of metals, compatible with the OWL process. Nonlimiting examples of metals contemplated include indium-tin-oxide, titanium, platinum, titanium tungstide, gold, silver, nickel, and copper. The polymer can be any polymer prepared by monomers that are polymerizable using electrochemistry. The polymer can be polypyrrole, a polyaniline, polythiophene, poly(ethylenedioxy)thiophene, compounds of poly(heteraromic vinylenes), polyvinylphosphate, and mixtures thereof. Optionally, the polymer can comprise an acceptable salt, e.g., tetrafluoroborate, and/or be doped with another polymer, e.g., poly(styrene p-sulfate). The polymer can be modified with optional substituents on an aryl ring of the corresponding monomer. Nonlimiting examples of such aryl substituents include, but are not limited to, cyano, sulfate, and nitro. Other suitable counterions, polymers for doping, and optional aryl ring substituents are well known to those of skill in the art.

Some ranges of length for the segment contemplated include about 10 nm to about 5 μm, about 10 nm to about 4 μm, about 10 nm to about 3 μm, about 10 nm to about 2 μm, about 10 nm to about 1 μm, about 10 nm to about 500 nm, and about 10 nm to about 400 nm. Specific examples of segment length include about 10, about 15, about 20, about 25, about 30, about 35, about 40, about 45, about 50, about 55, about 60, about 65, about 75, about 80, about 85, about 90, about 95, about 100, about 105, about 110, about 115, about 120, about 125, about 130, about 135, about 140, about 145, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400 nm, about 410 nm, about 420 nm, about 430 nm, about 440 nm, about 450 nm, about 460 nm, about 470 nm, about 480 nm, about 490 nm, about 500 nm, about 510 nm, about 520 nm, about 530 nm, about 540 nm, about 550 nm, about 560 nm, about 570 nm, about 580 nm, about 590 nm, about 600 nm, about 610 nm, about 620 nm, about 630 nm, about 640 nm, about 650 nm, about 660 nm, about 670 nm, about 680 nm, about 690 nm, about 700 nm, about 710 nm, about 720 nm, about 730 nm, about 740 nm, about 750 nm, about 760 nm, about 770 nm, about 780 nm, about 790 nm, about 800 nm, about 810 nm, about 820 nm, about 830 nm, about 840 nm, about 850 nm, about 860 nm, about 870 nm, about 880 nm, about 890 nm, about 900 nm, about 910 nm, about 920 nm, about 930 nm, about 940 nm, about 950 nm, about 960 nm, about 970 nm, about 980 nm, about 990 nm, and about 1 μm.

The gaps of the nanowires disclosed herein can be of any length, up to about 1 μm. In certain cases, the gap is about 2 to about 10 nm, and is designed by placement of a sacrificial metal segment during the OWL process. The gap can be about 2, about 3, about 4, about 5, about 6, about 7, about 8, about 9, or about 10 nm. The nanowire can contain a plurality of metal segments and gaps along the length of the nanowire. In some cases, the nanowire has only two segments separated by a single gap, while in other cases, the nanowire has two, three, four, five, or more gaps, and a corresponding number of segments (e.g., n+1).

The coating of the nanowire is a material that is positioned along one side of the nanowire. The purpose of the coating is to provide a bridging substrate to hold segments of the nanowire together. A nonlimiting example of a coating is silica.

The compounds spanning the gaps of the nanowires disclosed herein have a general formula (I)

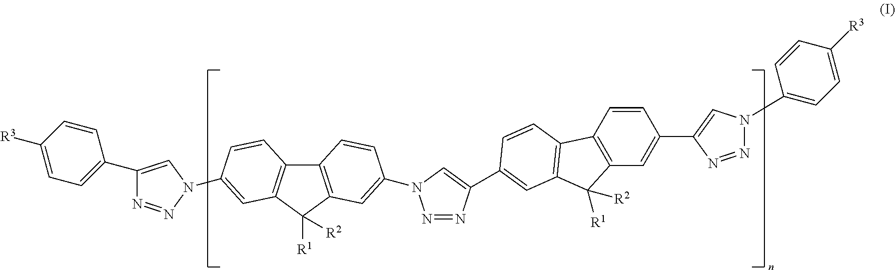

wherein R¹ and R² are independently selected from the group consisting of hydrogen, C₁-C₂₀alkyl, and C₁-C₂₀alkylenearyl, R³ is a moiety or residue of a moiety capable of forming a covalent bond or non-covalent interaction with a segment of the nanowire; and n is an integer of 1 to 20.

The compound of formula (I) can be prepared be admixing the nanowire and compounds of formulae (II), (III), and (IV) in the presence of a copper (I) salt:

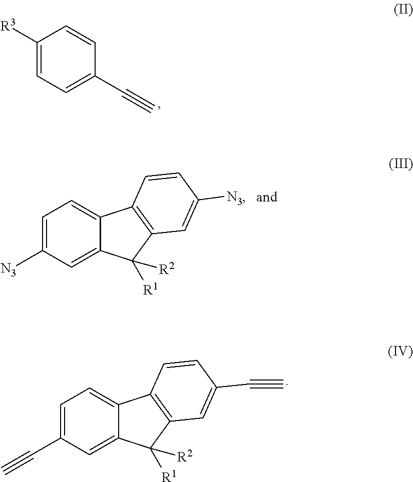

In some cases, the copper (I) salt is produced in situ by addition of a copper (II) salt and a reducing agent. Reducing agents are described in greater detail below.

As used herein, the term “alkyl” refers to straight chained and branched hydrocarbon groups, nonlimiting examples of which include methyl, ethyl, and straight chain and branched propyl and butyl groups. The term “alkyl” includes “bridged alkyl,” i.e., a bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. Alkyl groups optionally can be substituted, for example, with hydroxy (OH), halo, aryl, heteroaryl, ester, carboxylic acid, amido, guanidine, and amino.

As used herein, the term “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to four groups independently selected from, for example, halo, alkyl, alkenyl, OCF₃, NO₂, CN, NC, OH, alkoxy, amino, CO₂H, CO₂alkyl, aryl, and heteroaryl. Exemplary aryl groups include, but are not limited to, phenyl, naphthyl, tetrahydronaphthyl, chlorophenyl, methylphenyl, methoxyphenyl, trifluoromethylphenyl, nitrophenyl, 2,4-methoxychlorophenyl, and the like.

As used herein, the term “heteroaryl” refers to a monocyclic or bicyclic ring system containing one or two aromatic rings and containing at least one nitrogen, oxygen, or sulfur atom in an aromatic ring. Unless otherwise indicated, a heteroaryl group can be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

R³ can be a moiety that is compatible with the segment of the nanowire so as to form a covalent or non-covalent interaction between R³ and the segment. For example, when the segment comprises gold, R³ can be a thiol (protected or unproteced) such that a covalent bond between the sulfur of the thiol and the segment forms upon interaction between the segment and R³. Other examples include carboxylic acids on aluminum copper, titanium dioxide, or platinum, aromatic rings on platinum, and sulfoxides on platinum. In some cases, both R³ moieties at either end of the compound of formula (I) comprise a group that can form a covalent bond with the segment, while in other cases, only one of the two R³ moieties can form a covalent bond. In certain cases, the interaction between the compound and the segment is a non-covalent interaction.

The nanowires can be connected to electrodes. The electrodes can comprise any metal capable of conducting electrons. Non-limiting examples include gold, chromium, platinum, palladium, nickel, titanium, and silver.

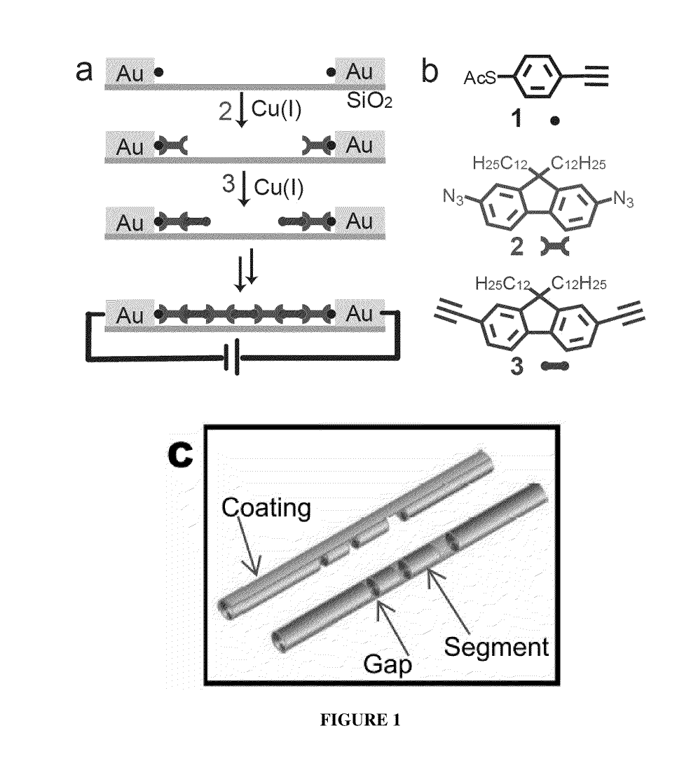

The general scheme for bridging the nanogaps with click chemistry for MTJ fabrication is shown in FIG. 1a. In an exemplary experiment, 4-ethynyl-1-thioacetylbenzene (compound 1) [19] is first assembled into a monolayer on the surfaces of the electrodes that are on opposite ends of an OWL-generated nanogap by immersing the entire device in a 1 mM dichloromethane/methanol (2/1, v/v) solution of compound 1 for 12 hr. Concentrated sulfuric acid (50 μL) is added in the solution for thiol deprotection.[20] The device is rinsed with dichloromethane/methanol, chloroform, and ethanol, respectively, and then immersed in a 10 mL dimethyl formamide (DMF) solution of a di-azido compound 2 [21] (1 mM) containing 200 μL of copper sulfate (0.074 M) and ascorbic acid (0.148 M). One of the azido groups of compound 2 reacts with the alkyne group on electrode-immobilized compound 1 to form a 1,2,3 triazole at one end and unreacted azide group at the other end. This structure can be further extended by reaction with 2,7-diethynyl-fluorene (compound 3),[21] which in turn can be reacted again with compound 2. Following the appropriate number of reaction cycles, the molecular wires growing from opposing electrodes combine and a compound of formula (I) bridges the nanogap. The point at which the bridge is formed can be determined from the I-V characteristics of the device, and the number of reaction steps required to form a bridge relates to the size of the gap.

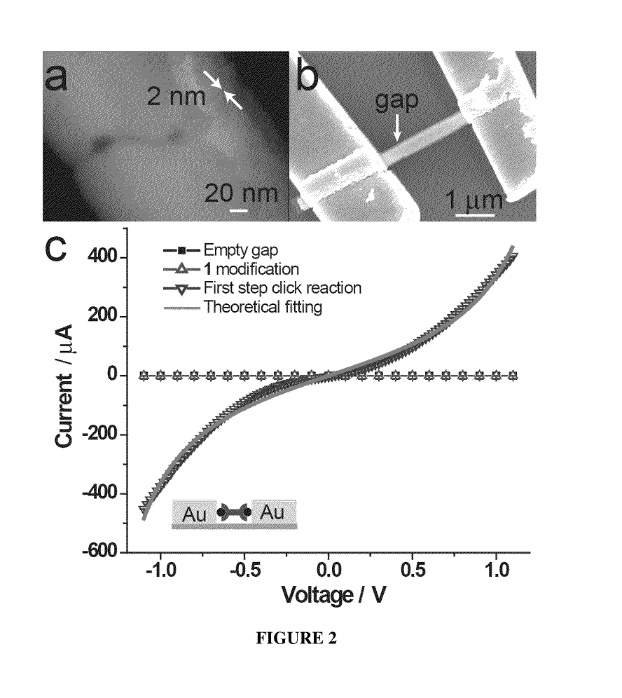

A one-step click reaction using compounds 1 and 2 to bridge a 2 nm OWL-generated gap and form an MTJ was performed. The calculated S—S distance of the target bridging molecule is 2.6 nm, which is sufficiently long enough to span the gap. In a typical experiment, 360 nm diameter wire structures with 2 nm nanogaps (FIG. 2a) were cast onto a substrate with gold microelectrodes and then connected to the electrodes by electron-beam lithography and subsequent chromium and gold thermal deposition (FIG. 2b). The two terminal I-V characteristics of the gap devices were measured at room temperature before and after click reactions (FIG. 2c). The empty nanogaps or nanogaps modified with only a monolayer of compound 1 exhibit no conductance within the noise limit of the measurement (<2 pA) (overlapping solid square and open uptriangle traces, FIG. 2c). However, following the click reaction of compounds 1 and 2 within the gap, I-V characteristics show a clear molecular response in the μA range (open downtriangle trace, FIG. 2c), which indicates the formation of a conjugated molecular bridge within the nanogap as a result of the click reaction.

The yield for working devices was 41% (12 out of 29 devices with I>0.1 nA at 1 V bias). It should be noted that the magnitude of the current measured with different devices varies from 0.1 nA to 600 μA at 1 V bias, which is theorized to result from different numbers of molecules bridging the nanogap in different experiments. It is also possible that the roughness of the electrode surface contributes to the observed variation. As a control experiment, the dithiol compound 4 was synthesized ex situ from compounds 1 and 2, and the current amplitude and yield of working devices was lower (about 10%, 3 out of 31) than that of MTJ devices assembled in situ. It is theorized that this observation may result from a slow diffusion of the large molecule into the nanogap. [11]

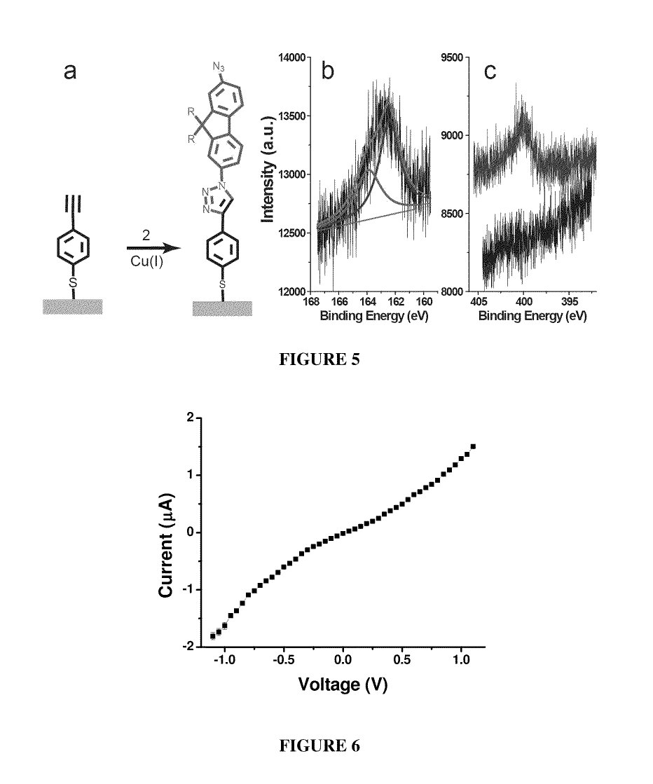

X-ray photoelecton spectroscopy (XPS) on a bulk surface confirmed that the click reactions proceeded on gold. For example, the assembly of compound 1 and the click reaction between compounds 1 and 2 were performed on a planar gold substrate surface and followed by XPS in the S 2p region and the N 1s region. In general, the S 2p spectra are composed of 2p3/2 and 2p1/2 peaks with an intensity ratio of 2:1, as theoretically determined from the spin-orbit splitting effect. FIG. 5 shows binding peaks at 162.4 (S 2p3/2) and 163.9 eV (S 2p1/2), which are assigned to the bound sulfur. [22] Furthermore, when a surface-bound monolayer of compound 1 reacts with compound 2 in the presence of Cu^{I}, a peak at 400.2 eV is observed, which arises from the presence of both triazoles and azides (N 1s). [23] These spectral signatures confirm that the 1,3-dipolar cycloaddition between azides and alkynes proceed successfully on monolayer-modified Au surfaces.

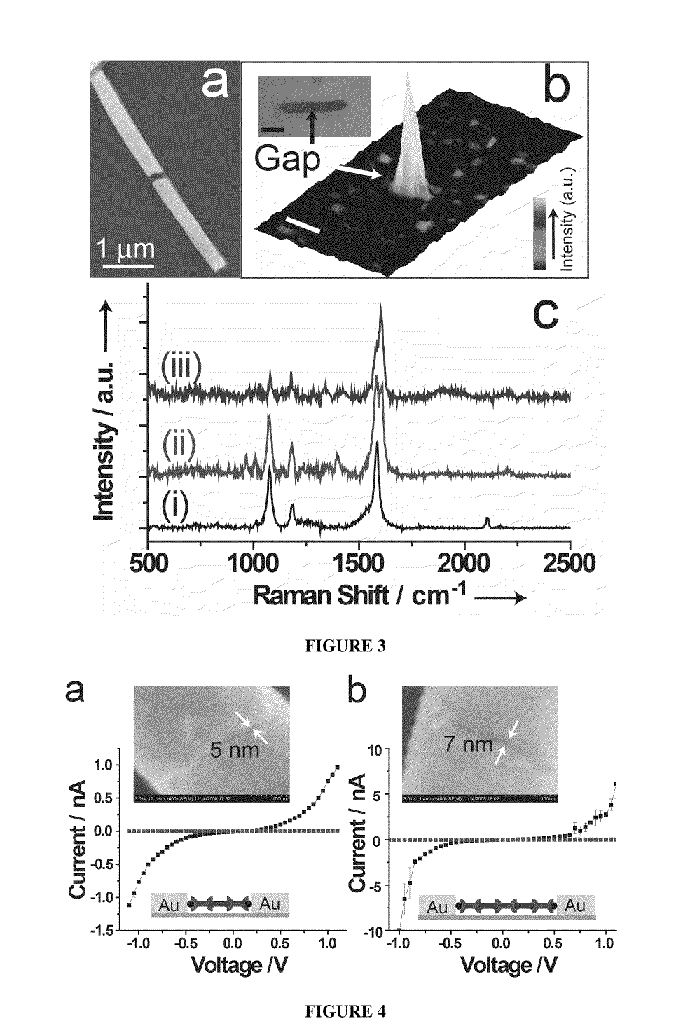

SERS measurements directly on the nanogaps confirm that the click reaction proceeds within this confined space. Sub-100 nm OWL-generated nanogaps have been shown to act as Raman “hot spots” with enhancement factors as large as 10^{8 [}9,11], such that molecules assembled within nanogaps can be efficiently identified by SERS.[24,25] To evaluate the potential of OWL-generated nanogaps for simultaneous assembly and spectroscopic identification, sub-100 nm OWL-generated nanogap structures with gold segments on the opposite sides of the nanogaps were fabricated. In a typical experiment, a nanogap (98±11 nm) (FIG. 3a) was used to assemble the molecules through click chemistry as described for the aforementioned MTJ fabrication, and the Raman spectra and image of the gap area were measured by a confocal scanning Raman microscopy (WiTec Alpha300). For gap structures modified with compound 1, the Raman spectrum ((i) of FIG. 3c) clearly shows the presence of alkyne groups (CC symmetric stretch at 2108 cm⁻¹) and phenyl groups (C—C benzene ring stretching of 1 at 1585 cm⁻¹). In addition, compared with the spectrum of the neat solid of compound 1, the absence of thioacetyl (634 cm⁻¹) and SH (bending, 915 cm⁻¹) vibration modes indicates Au—S bonding.[26] The confocal Raman images (FIG. 3b) obtained by integrating the spectral intensity from 1520 to 1620 cm⁻¹ and the bright-field optical image shows the hot spots are localized in the gap. When compound 2 reacts with the monolayer of compound 1 through click chemistry, new peaks at 967 and 1010 cm⁻¹ (the triazole ring stretch) [27], 1606 cm⁻¹ (C—C benzene ring stretching of compound 2), and 2198 cm⁻¹ (asymmetric stretching of azide groups) appear, and the peak at 2108 cm⁻¹ disappears ((ii) of FIG. 3c), indicating the click reaction of compounds 1 and 2 proceeded. Furthermore, when compound 3 reacts with compound 2 through click chemistry, the relative intensity of the peak at 1606 cm⁻¹ increases ((iii) of FIG. 3c), which confirms the occurrence of the reaction between compounds 3 and 2. These SERS experiments demonstrate the ability to directly observe chemical reactions within nanogaps and, as a result, confirm the chemical composition of the molecules present within the MTJs.

Theoretical calculations were performed to characterize the transport behavior observed across the MTJs. Density functional theory calculations (B3LYP, 6-31G*) were carried out on the gas-phase (geometry optimized) molecular wire, and the HOMO-LUMO gap was determined to be 3.8 eV (HOMO: −5.6 eV, LUMO: −1.8 eV). In addition, a single level model [28] was used to fit the experimental I-V curve. Assuming transport is in the Landauer regime of coherent tunneling [29-31], a transport equation dominated by one channel (single level picture) was formulated to obtain the electrode-molecule coupling (0.037 eV on both sides) and the energy gap between the Fermi level and the molecular level (0.75 eV). By taking a gold Fermi level around 5 eV, these results indicate that hole transport (i.e., through the HOMO) dominates. The experimental results (open downtriangle curve, FIG. 2c) and theoretical fit (FIG. 2c) are in good agreement.

Multistep reactions also can be carried out within the nanogaps in a stepwise approach analogous to solid-phase synthesis. The ability to extend and vary chemical structure within MTJs in situ allows the determination, in a high throughput, combinatorial fashion, of how changes in molecular structure within the gaps affect transport properties. To this end, compound 3 was synthesized, a fluorene derivative having two alkyne substituents. When compound 3 is reacted with the available azide from compound 2 on a gold electrode, under click reaction conditions, the oligomeric fluorene chain is extended. This either bridges the gap or leaves an available azide for further reaction (FIG. 4). The number of fluorene monomers in the oligomeric chain is controlled precisely by the number of reaction steps and the gap size. MTJs with three and five fluorene monomers have been synthesized from two and three click reaction steps, in 5 and 7 nm gaps, respectively. Transport is observed only after the appropriate number of reaction steps to bridge the gap have been performed. In the case of the 5 nm gap, no current is observed until two reactions have been performed, and in the case of the 7 nm gap, until after three reactions. These experiments demonstrate the ability to carry out multiple reaction steps within the gap.

A new method for the in situ, modular fabrication of MTJs through click chemistry in OWL-generated nanogaps is disclosed herein, whereby the Raman enhancement inherent to these nanostructures is used to spectroscopically characterize the molecular assembly processes within the gaps. The use of click chemistry to form MTJs proceeds in high yields and can be used to test different molecules, and the triazole form maintains conjugation in the molecular wires. In addition, this method overcomes a major challenge in the field of molecular electronics: i.e., an ability to spectroscopically track the assembly processes of MTJs within such tiny gaps. By using the azide-alkyne click reaction to affix molecules within the gap, the transport properties of different functional building blocks can be explored. This concept was demonstrated by synthesizing fluorene oligomers of different lengths within the gaps and studying their transport. This method of forming and characterizing MTJs can be used to create nanoelectronic devices with diverse functions and applications.

### Copper Detection

Click chemistry can also be used as a means to detect copper (and measure copper concentrations) in a sample, because a Cu(I) catalyst is needed to form an azole from an alkyne and an azide. Detection of copper is important because copper at elevated concentrations can be highly toxic to organisms such as algae, fungi and many bacteria, and may cause adverse health effects to the gastrointestinal, hepatic and renal systems.[35,36] As such, the detection and measurement of copper has become increasingly important, especially in an on-site format. Several methods exist for the detection of Cu²⁺ ions, including those based on organic dyes, semiconductor nanocrystals, spectroscopy, and cyclic voltammetry.[37-43] The read out of these methods, however, require sophisticated instrumentation. Colorimetric methods are extremely attractive for on-site use because they can be easily read without the aid of sophisticated instrumentation.

As used herein, the term “sample” refers to biological and environmental samples. Biological samples include, but are not limited to, a fluid, such as urine, blood, plasma, serum, saliva, semen, stool, sputum, cerebral spinal fluid, tears, mucus, and the like. Biological samples can be from human or animal. Environmental samples include, but are not limited to, soil and water, such as groundwater.

Oligonucleotide gold nanoparticle (Au NP) conjugates [44,45] have been used in a variety of formats to detect oligonucleotides, proteins, metal ions, enzyme inhibitors,[46-53] and intracellular mRNA.[54] Oligonucleotide Au NPs have high extinction coefficients (about 4 orders of magnitude greater than typical organic dyes) and unique distance dependent optical properties. When hybridized to complementary particles, oligonucleotide Au NPs turn from red to purple, and their aggregates exhibit extremely sharp melting transitions which make them useful for the colorimetric detection of oligonucleotides. These optical and melting properties have also been utilized in mercuric ion detection, where deliberately designed T-T mismatches in the interparticle oligonucleotide duplexes bind that ion via coordination chemistry. The presence of the T-Hg²⁺-T coordination complexes raise the melting temperature (Tₘ) of the polymeric aggregates, such that the solution color at a given temperature can be used to determine Hg²⁺ concentration. [49,53]

Recently, gold nanoparticle-based colorimetric detection approaches for copper ions have been developed.[55,56] However, these systems require labile leaving groups or long incubation times which limit their utility in on-site applications. The method disclosed herein uses a colorimetric method for the detection of Cu²⁺ ions based on densely functionalized oligonucleotide nanoparticle (NP) conjugates and a copper-catalyzed azide-alkyne cycloaddition (click) reaction. This new approach relies on the ligation of two oligonucleotide strands within polymeric aggregates of the oligonucleotide NPs. The oligonucleotide strands are terminated with an alkynyl (e.g., a hexynyl) group or azide group, allowing them to be ligated, after reaction between the alkyne and azide, into a longer oligonucleotide strand. The use of click chemistry and oligonucleotide NPs confers several advantages. First, they use the oligonucleotides as a template to align the alkyne and azide groups for optimal reactivity.[57, 58] Second, the sharp melting properties of the oligonucleotide Au NPs allow one to distinguish a subtle difference in Tₘ, which is preferred for this assay.[59] Finally, the alkyne and azide groups are robust, and their cyclization is catalyzed specifically by copper ions.[60,61] This ligation can raise the Tₘ of the aggregate in proportion to the amount of copper ion present, which can be monitored by UV Vis spectroscopy or the naked eye.

In a typical experiment, two sets of oligonucleotide Au NP conjugates were prepared by functionalizing two batches of 30 nm Au NPs with different thiol-modified oligonucleotide strands. The first type of particle, called the template particle, was modified at one terminus with a thiol group, e.g., 3′-propylthiol (for example, SEQ ID NO: 1: 5′ TAG GAA TAG TTA TAA GCG TAA GTC CTA ACG A₁₀ SH 3′). The second particle, the alkyne Au NP, was functionalized with a thiol group at one terminus and an alkyne at the other terminus, e.g., 3′-propylthiol and 5′-alkylated, and comprised a sequence for the oligonucleotide and was complementary to at least a portion of half of the oligonucleotide on the template particles (for example, SEQ ID NO: 2: 5′ hexynyl TTA TAA CTA TTC CTA A₁₀ SH 3′). A mixture of these functionalized Au NPs form polymeric networks upon mixing due to their complementary DNA and resulting hybridization.

A third oligonucleotide, the azide strand, is added to the mixture of SEQ ID NO: 1 and 2. This strand is has an azide group at one terminus, e.g., a 3′ azidobutyrate (for example, SEQ ID NO: 3: 5′ CGT TAG GAC TTA CGC azidobutyrate 3′) and is complementary to the other half of the template strand (e.g., SEQ ID NO: 1).

While the examples described herein refer to gold nanoparticles, the nanoparticle can comprise any metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Other nanoparticles useful in the practice of the invention include, also without limitation, ZnS, ZnO, Ti, TiO₂, Sn, SnO₂, Si, SiO₂, Fe, Ag, Cu, Ni, Al, steel, cobalt-chrome alloys, Cd, titanium alloys, AgI, AgBr, HgI₂, PbS, PbSe, ZnTe, CdTe, In₂S₃, In₂Se₃, Cd₃P₂, Cd₃As₂, InAs, and GaAs. Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshaysky, et al., J. Am. Chem. Soc., 112, 9438 (1990); and Ushida et al., J. Phys. Chem., 95, 5382 (1992).

In practice, methods are provided using any suitable nanoparticle having oligonucleotides attached thereto having the suitable characteristics described herein, and that are in general suitable for use in the disclosed detection assays, which do not interfere with oligonucleotide complex formation, i.e., hybridization. The size, shape and chemical composition of the particles contribute to the properties of the resulting oligonucleotide-functionalized nanoparticle. These properties include for example, optical properties, optoelectronic properties, electrochemical properties, electronic properties, stability in various solutions, magnetic properties, and pore and channel size variation. The use of mixtures of particles having different sizes, shapes and/or chemical compositions, as well as the use of nanoparticles having uniform sizes, shapes and chemical composition, is contemplated. Examples of suitable particles include, without limitation, nanoparticles, aggregate particles, isotropic (such as spherical particles) and anisotropic particles (such as non-spherical rods, tetrahedral, prisms) and core-shell particles, such as those described in U.S. Pat. No. 7,238,472 and International Publication No. WO 2003/08539, the disclosures of which are incorporated by reference in their entirety.

The nanoparticle can have a diameter of about 10 nm to about 1 μm, or about 10 nm to about 500 nm, about 10 nm to about 400 nm, about 10 nm to about 300 nm, about 10 nm to about 200 nm, about 10 nm to about 100 nm, or about 10 nm to about 50 nm. Specific contemplated diameters include about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 55, about 60, about 65, about 70, about 75, about 80, about 85, about 90, about 95, about 100, about 110, about 120, about 130, about 140, about 150, about 160, about 170, about 180, about 190, or about 200 nm.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles is described in Fattal, et al., J. Controlled Release (1998) 53: 137-143 and U.S. Pat. No. 4,489,055. Methods for making nanoparticles comprising poly(D-glucaramidoamine)s are described in Liu, et al., J. Am. Chem. Soc. (2004) 126:7422-7423. Preparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers). Suitable nanoparticles are also commercially available from, for example, Ted Pella, Inc. (gold), Amersham Corporation (gold) and Nanoprobes, Inc. (gold). Tin oxide nanoparticles having a dispersed aggregate particle size of about 140 nm are available commercially from Vacuum Metallurgical Co., Ltd. of Chiba, Japan. Other commercially available nanoparticles of various compositions and size ranges are available, for example, from Vector Laboratories, Inc. of Burlingame, Calif.

Also, as described in U.S. patent publication No 2003/0147966, nanoparticles comprising materials described herein are available commercially, or they can be produced from progressive nucleation in solution (e.g., by colloid reaction) or by various physical and chemical vapor deposition processes, such as sputter deposition. See, e.g., HaVashi, Vac. Sci. Technol. A5(4):1375-84 (1987); Hayashi, Physics Today, 44-60 (1987); MRS Bulletin, January 1990, 16-47. As further described in U.S. patent publication No 2003/0147966, nanoparticles contemplated are produced using HAuCl₄ and a citrate-reducing agent, using methods known in the art. See, e.g., Marinakos et al., Adv. Mater. 11:34-37 (1999); Marinakos et al., Chem. Mater. 10: 1214-19 (1998); Enustun & Turkevich, J. Am. Chem. Soc. 85: 3317 (1963).

The first oligonucleotide, the “template” oligonucleotide, is attached to a first nanoparticle (such as through a thiol bond to a gold nanoparticle), and comprises about 15 to about 100 nucleobases. The template oligonucleotide can be about 20 to about 70, about 22 to about 60, or about 25 to about 50 nucleobases.

The second oligonucleotide, the “alkyne” oligonucleotide, is attached to a second nanoparticle (such as through a thiol bond to a gold nanoparticle), comprises about 5 to about 50 nucleobases and an alkyne moiety at one terminus (either the 3′ or 5′ terminus). The sequence of the second oligonucleotide is sufficiently complementary to at least a portion of the sequence of the first oligonucleotide to permit hybridization therewith. The portion of the template oligonucleotide and alkyne oligonucleotide are typically at least about 50% complementary along that portion, but can be at least about 60%, at least about 70%, at least about 80%, or at least about 90% complementary along that portion. The alkyne oligonucleotide can be about 10 to about 45, about 15 to about 40, or about 15 to about 30 nucleobases.

The third oligonucleotide, the “azide” oligonucleotide, is not attached to a nanoparticle, comprises about 5 to 50 nucleobases and an azide moiety at one terminus (either the 3′ or 5′ terminus, the opposite terminus compared to the terminus modified in the alkyne oligonucleotide). The sequence of the third oligonucleotide is sufficiently complementary to at least a portion of the sequence of the first oligonucleotide to permit hybridization therewith. The template and azide oligonucleotides are complementary for at least a portion of the template oligonucleotide and are typically at least about 50% complementary along that portion, but can be at least about 60%, at least about 70%, at least about 80%, or at least about 90% complementary along that portion. The azide oligonucleotide can be about 10 to about 45, about 15 to about 40, or about 15 to about 30 nucleobases.

In some embodiments, the alkyne nucleotide is not attached to a nanoparticle and the azide oligonucleotide is attached to the first nanoparticle.

Methods of making oligonucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically. Non-naturally occurring nucleobases can be incorporated into the oligonucleotide, as well. See, e.g., Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002). Introduction of an azide or alkyne moiety to the oligonucleotides disclosed herein can be through synthetic means well within the skill of the synthetic chemist.

Together, the three oligonucleotides form three stranded aggregates with a concomitant red-to-purple (or to colorless) color change upon aggregation. Next, the copper (II) concentration was increased to 200 μM with copper (II) sulfate, and excess sodium ascorbate (a reducing agent) and a water soluble tris-triazolylamine Cu(I) binding ligand [57,62] were added to the aggregates solution and incubated for 2 hours to allow for the click-chemistry ligation to occur. After click chemistry ligation, the aggregates had a colorless or light purple color.

The presence of copper results in a higher melting temperature of the hybridized oligonucleotides, which can be spectroscopically, and in certain cases, visually, detected. Absorbance of the nanoparticles can be monitored at 525 nm, where gold nanoparticles have maximum intensity. The absorbance is decreased when the nanoparticles are hybridized to other nanoparticles. When the oligonucleotides melt, an increase in absorbance results. This melting can also be seen as a color change from colorless or pale purple to a dark red when the hybridized oligonucleotides on the functional nanoparticles melt. Thus, the presence of copper increases the melting temperature of the nanoparticles, and development of color (from colorless for click chemistry ligated hybridized oligonucleotides on the nanoparticles to purple to red for de-hybridized oligonucleotides) does not occur until higher temperatures. The change in melting temperature can be correlated to the concentration of the copper. Thus, a comparison of the melting temperature of a sample having copper of unknown concentration to a standard curve of melting temperatures of known concentration of copper can provide the concentration of the copper in the sample.

The melting temperature, which was monitored at the Au NP surface plasmon resonance maximum of 525 nm, was 62.6° C. In a control experiment performed in the absence of copper ion, the aggregates melt at 50.4° C., approximately 12° C. below the Tₘ after ligation (FIG. 11). This increase in Tₘ is due to the conversion of the three-strand 30 bp nicked structure to a ligated two-strand 30 bp duplex. The difference in melting temperature can be at least 3° C., 3° C. to 20° C., 3° C. to 15° C., or 4° C. to 10° C.

The limit of detection of the assay was 20 μM and dynamic range is 20 μM to about 100 μM copper. The U.S. Environmental Protection Agency defines maximum contaminant level for copper in drinking water is 20 μM, making this assay relevant for testing drinking water.

Other contemplated reducing agents for use in the methods disclosed herein include, but are not limited to, ascorbic acid, an ascorbate salt, sodium borohydride, 2-mercaptoethanol, dithiothreitol (DTT), hydrazine, lithium aluminum hydride, diisobutylaluminum hydride, oxalic acid, Lindlar catalyst, sulfite compounds, stannous compounds, ferrous compounds, sodium amalgam, tris(2-carboxyethyl)phosphine, hydroquinone, and the like.

Other contemplated copper ligands for use in the methods disclosed herein include substituted tris-triazolamines, bathophenanthroline disulfonic acid, N,N,N′,N′,N″-pentamethyldiethylenetriamine, N,N-dimethylcyclohexane-1,2-diamine, N,N,N-trimethylethane-1,2-diamine, substituted bipyridines, and 1,10-phenanthroline.

In various cases, the methods disclosed herein can be used to determine the concentration of copper in a sample. The change in melting temperature can be correlated to the concentration of the copper. Thus, a comparison of the melting temperature of a sample having an unknown concentration of copper to a standard curve of melting temperatures of known concentrations of copper can provide the concentration of the copper in the sample.

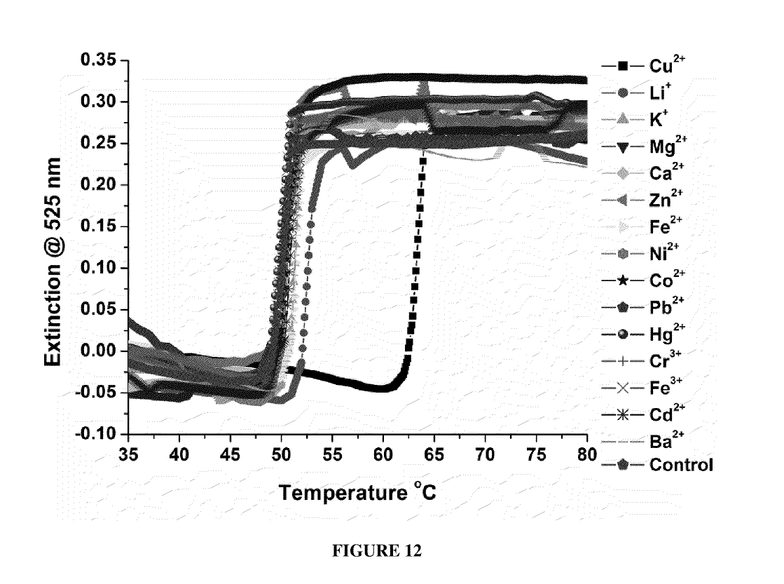

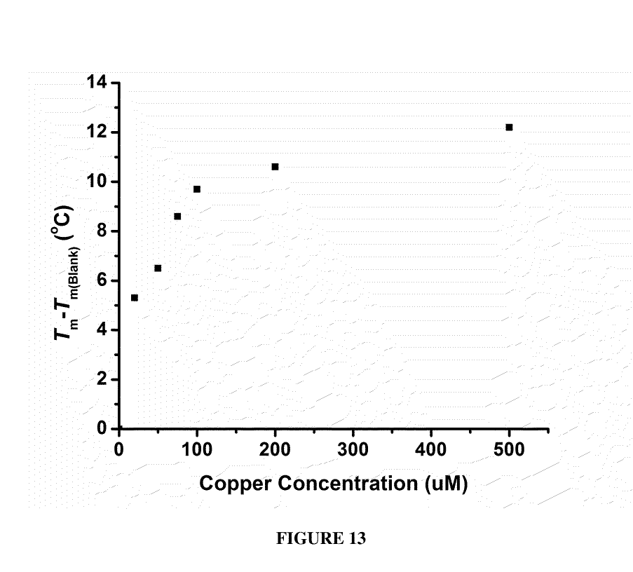

To evaluate the selectivity of this assay, the assay was performed in the presence of various metal ions, including Li⁺, K⁺, Mg²⁺, Ca²⁺, Fe²⁺, Mn²⁺, Co²⁺, Zn²⁺, Ni²⁺, Ba²⁺, Cd²⁺, Pb²⁺, Hg²⁺, Cr³⁺ and Fe³⁺. In a typical experiment, one of these metal ions was added to solutions of the DNA Au NP aggregates at a final concentration of 200 μM and incubated for 2 hours the presence of the tris-triazolylamine Cu⁺-binding ligand and sodium ascorbate. Only the Cu²⁺ sample shows an increased melting temperature (ΔTₘ=12° C.) relative to the blank (FIG. 12). No detectable melting temperature increase was observed for other metal ions. The optical properties of the DNA Au NPs allow copper ion detection through visual inspection.

A colorimetric copper ion detection system with high selectivity and sensitivity is disclosed herein. The concentration of Cu²⁺ can be determined by the change in solution color at a given temperature, or though a measurement of the melting temperature of the DNA Au NP aggregates. In contrast to other Au NP based detection systems, this method does not require labile reactive groups or long incubation times due to the robustness of the alkyne and azide functionalities and the fact that those groups are templated together via oligonucleotide hybridization, respectively. Taken together, these advantages make this assay simple, robust, inexpensive, and therefore useful for on-site water testing.

The invention will be more fully understood by reference to the following examples which detail exemplary embodiments of the invention. They should not, however, be construed as limiting the scope of the invention. All citations throughout the disclosure are hereby expressly incorporated by reference.

### EXAMPLES

### General Materials and Methods

Unless otherwise noted, all chemicals and reagents used in this study were purchased from commercial sources and used as received. Compounds 1, 2, and 3 were prepared according to literature procedures.[32,33] ¹H and ¹³C NMR spectra used in the characterization of the products were carried out on Varian INOVA 300 MHz or Bruker Avance III 600 MHz spectrometers using the residual solvent proton as the reference. Electrospray mass spectra were obtained on a Micromass Q-tof Ultra TOF MS operating in positive ion mode. Nanogaps were fabricated by On-Wire Lithography as described previously.[34] Scanning electron microscopy (Hitachi S4800) was used for morphology measurements. Raman spectra were recorded with a confocal Raman microscope (Alpha300 WiTec) equipped with a piezo scanner and 100× microscope objectives (n.a.=0.90; Nikon, Tokyo, Japan). Samples were excited with a 632.8 nm He—Ne laser (Coherent, Inc., Santa Clara, Calif.) with a power density of ˜10⁴ W/cm², with the long asix of the nanowires parallel to the laser polarization.

### Synthesis of Compound 4

Synthesis of compound 4 is outlined in the following scheme.

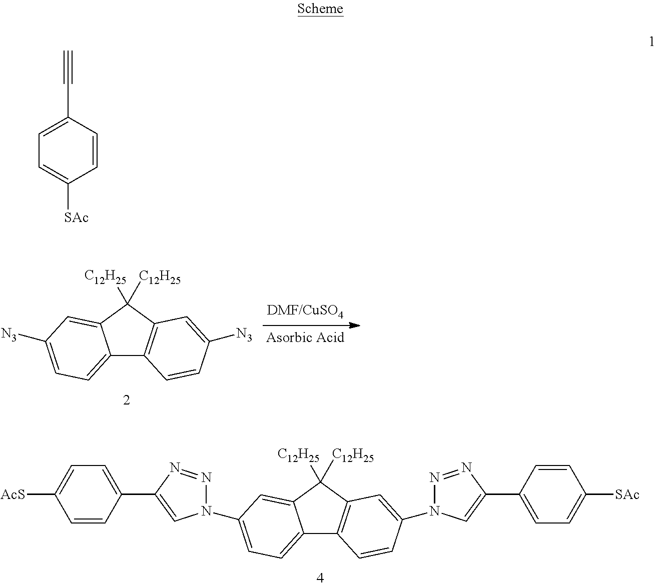

CuSO₄ (3.0 mg, 0.019 mmol) and ascorbic acid (3.0 mg, 0.017 mmol) were degassed in DMF for 20 min under an inert nitrogen atmosphere. 4-ethynyl-1-thioacetylbenzene (25 mg, 0.14 mmol) and 2,7-di-azido-fluorene (2) (41 mg, 0.070 mmol) were added to the reaction solution and stirred for 16 h. The solvent was removed in vacuo, and the mixture was purified by column chromatography (SiO₂:2:1 Hexane:CH₂Cl₂) to afford 4 (11 mg, 17%). ¹H NMR, 300 MHz, CDCl₃: δ 8.28 (s, 2H); 7.98 (d, 4H, J=2 Hz); 7.78 (d, 2H, J=3 Hz); 7.72 (d, 2H, J=3 Hz); 7.53 (d, 4H, J=2 Hz); 7.00 (s, 2H); 2.46 (s, 3H); 2.05-2.00 (m, 4H); 1.27-1.05 (m, 36H); 0.85 (t, 6H, J=2 Hz); 0.63 (s, 4H). ¹³C NMR, 150 MHz, CDCl₃: δ 194.14; 153.43; 152.71; 141.38; 137.13; 135.19; 131.68; 128.19; 126.77; 121.52; 120.61; 99.63; 118.35; 115.50; 113.96; 56.10; 40.58; 32.12; 30.51; 30.15; 29.83; 29.78; 29.54; 25.52; 24.01; 22.90; 14.34. MS (ESI⁺) calcd for C₆₄H₈₇O₃S₂ [M+MeOH+H⁺]: 967.6097; found: 967.5513.

### Photolithography and E-Beam Lithography

Silicon wafers with a 600 nm of oxide layer were cleaned by sonicating in acetone and ethanol for 30 min. They were subsequently rinsed with ethanol and dried with N₂. A photoresist (AZ 1518 Photoresist, Shipley, USA) was applied to the wafer, which was subsequently spun at 3000 rpm for 30 s. After the spin coating, the wafers were put in an oven (90° C.) for 30 min. The resist was patterned using a mask aligner (Q-2000 Quintel Mask Aligner, San Jose, Calif., USA) and developed with AZ 300 MIF. Then, Cr (5 nm) and Au (50 nm) were thermally evaporated onto the patterned wafer. Finally, the microscopic electrodes were formed after the patterned wafer was immersed in acetone for liftoff.

One drop of an aqueous solution of nanorods with OWL-fabricated nanogaps was deposited on a chip containing prefabricated Au electrodes, and the chip was dried in vacuum. Electron beam lithography (EBL) was utilized to define an inner electrode pattern that connected the nanorods with the microelectrodes. A resist layer of poly(methyl methacrylate) (PMMA) was prepared by the following procedure: 950 PMMA C7 was spincoated at 500 rpm (10 second) and 3000 rpm (45 second) followed by baking at 180° C. for 2 min. EBL was carried out using a FEI Quanta FESEM equipped with the Nabity Pattern Generation System (NPGS, JC Nabity Lithography System, Bozeman, Mont., USA) at 30 kV acceleration voltage and 30 pA beam current. Cr (7 nm) and Au (400 nm) were then thermally evaporated onto the e-beam resist-coated substrate after it had been developed with 3:1 (v/v) isopropyl alcohol/methyl isobutyl ketone (IPA/MIBK) solution for 1 min, and then rinsed with IPA and water.

### Electrical Measurements

The electrodes were wire-bonded to a chip carrier using a wedge wire-bonder (K&S 4526 wire-bonder, Kulicke & Soffa, Willow Grove, Pa., USA). The current-voltage characteristics of the devices were then obtained using a shielded, temperature-controlled cryostat (Optistat CF-continuous flow, exchange gas cryostat, Oxford Instruments, Oxford, UK) equipped with coaxial connections. All of the measurements were made in the absence of light. For all measurements, a 16-bit digital acquisition board (DAQ, National Instruments, Austin, Tex.) and preamplifier (Model 1211, DL Instrument, Ithaca, N.Y., USA) were used for the voltage source and current measurements.

### Theoretical Model

The theoretic model begins with the Hamiltonian for a single molecular level (at energy γ₀ with creation/annihilation operators c₀⁺/c₀) coupled to left and right electrodes (at energies γₖ with creation/annihilation operators cₖ⁺/cₖ) with coupling strength Vₖ:

$$H = {ɛ}_{0} {c}_{0}^{+} {c}_{0} + {\sum }_{k ∈ \{ L , R \}} {ɛ}_{k} {c}_{k}^{+} {c}_{k} + {\sum }_{k ∈ \{ L , R \}} {V}_{k} {c}_{k}^{+} {c}_{0} + h . c .$$

This describes a simple model for the molecular junction. Within the Landauer approximation, the current, I, through the junction is given by:

$$I = \frac{2 e}{ℏ} {\int }_{- ∞}^{∞} \frac{d E}{2 π} ( {f}_{L} ( E ) - {f}_{R} ( E ) ) {\vert {G}^{r} ( E ) \vert}^{2} {Γ}_{L} {Γ}_{R}$$

where f_{L,R} are the voltage-dependent Fermi functions, [symbol] _{L,R} are the electrode-molecule couplings (taken as constants in the wide-band limit), and G^{r} is the retarded Green's function given by

G^{r}(E)=[E−ε₀+i(Γ_{L}+Γ_{R})]⁻¹.

Unknown are the quantities of γ₀ and [symbol] _{L,R}. By fitting the experimental current-voltage plots to our theoretical equation, one can deduce these values. The model is symmetric such that an identical fit is obtained for ∀γ₀. To identify the dominant transport channel, γ₀ is identified as the energy gap between the electrode Fermi level and either the HOMO or LUMO of the molecule. Taking this Fermi level to be 5.1 eV leads to a conclusion that transport is through the HOMO for these molecules.

### XPS Measurements

Samples were transferred to an analysis chamber equipped with an X-ray photoelectron spectrometer (XPS) (Omicron). An Al Kα (1486.5 eV) anode with a power of 200 W (20 KV) was used for all measurements. XPS spectra were obtained using a hemispherical energy analyzer operated at a pass energy of 20.0 eV. Binding energies were referenced to the Au4f peak at 84.0 eV for pure gold.

Control Experiment: Assembly of Compound 4 within a 2 nm Gap

Compound 4 was assembled within a 2 nm nanogap to bridge the gap, where 50 μL of ammonium hydroxide (NH₄OH) was added in the solution for thiol deprotection. I-V curves were measured on the resulting MTJs, and both device yield as well as current amplitude decrease when compared to the in situ fabricated molecular wires (FIG. 6).

### Raman Spectra of the Neat Bulk Sample of Compounds 1, 3, and 4

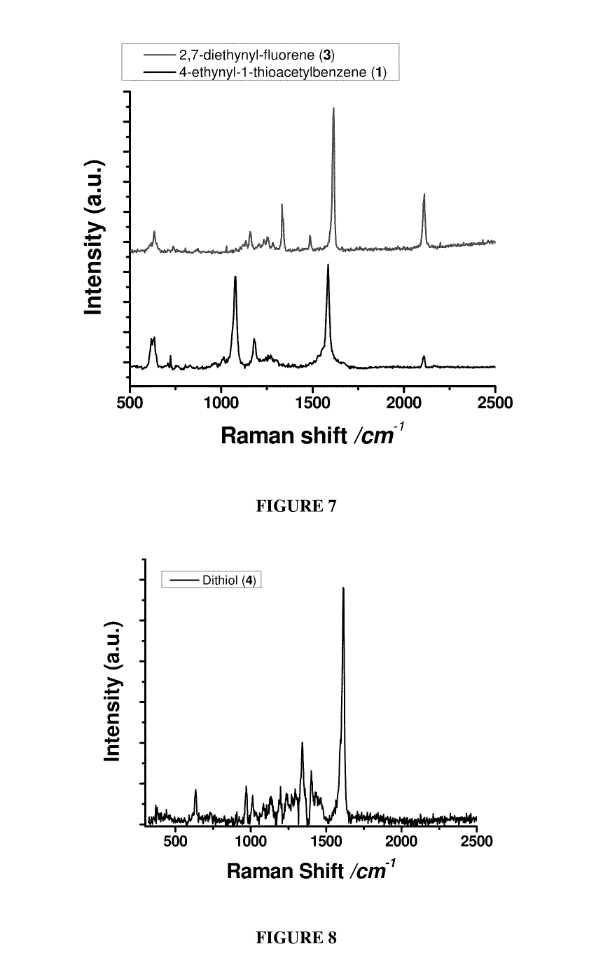

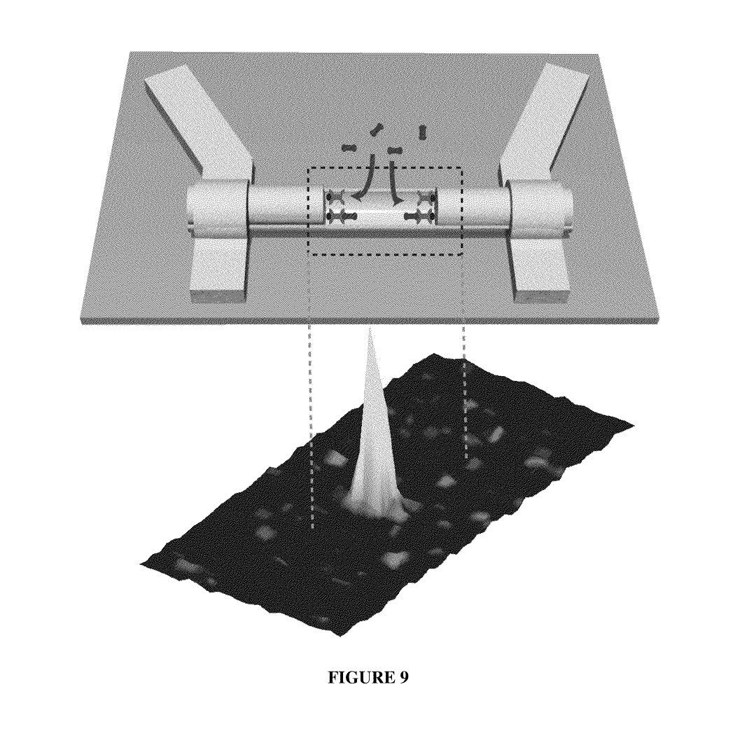

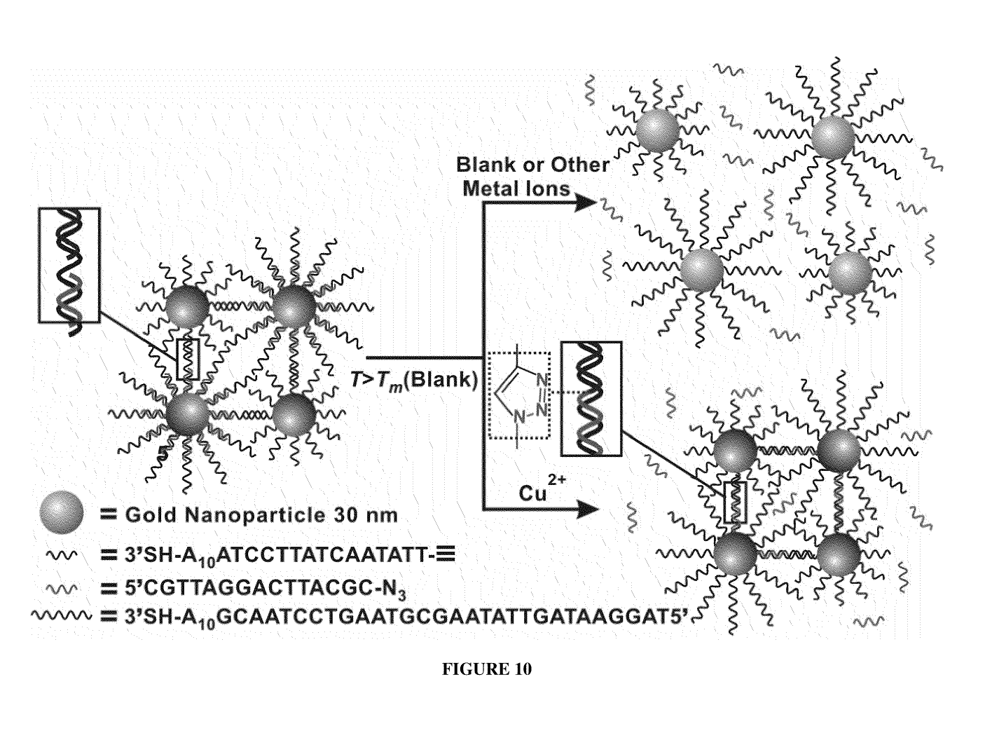

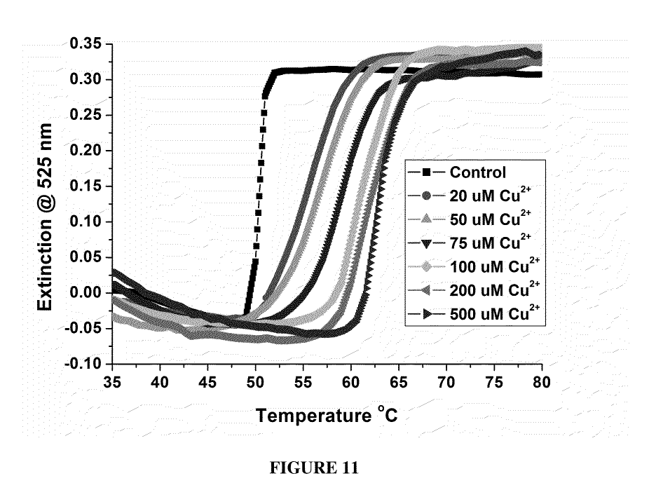

The Raman spectra of neat, bulk samples of compounds 1, 3, and 4 were taken for comparison with the SERS spectra of the deprotected molecules taken within the nanogaps. These bulk spectra are in good agreement for the SERS spectra taken within the nanogaps. FIG. 7 (top) shows the Raman spectrum of compound 3; FIG. 7 (bottom) shows the Raman spectrum of compound 1; and FIG. 8 shows the Raman spectrum of compound 4.

While the present invention has been described in terms of various embodiments and examples, it is understood that variations and improvements will occur to those skilled in the art. Therefore, only such limitations as appear in the claims should be placed on the invention.

### REFERENCES

[1] Nitzan, et al, Science 2003, 300, 1384-1389.
[2] Tao, Nat. Nanotechnol. 2006, 1, 173-181.
[3] Tour, Acc. Chem. Res. 2000, 33, 791-804.
[4] Taniguchi, et al., J. Am. Chem. Soc. 2006, 128, 15062-15063.
[5] Tang, et al., Angew. Chem. Int. Ed. 2007, 46, 3892-3895.
[6] Qin, et al., Science 2005, 309, 113-115.
[7] Chen, et al., J. Am. Chem. Soc. 2008, 130, 8166-8168.
[8] Qin, et al., Small 2007, 3, 86-90.
[9] Qin, et al., Proc. Natl. Acad. Sci. U.S.A. 2006, 103, 13300-13303.
[10] Qin, et al., Nano Lett. 2007, 7, 3849-3853.
[11] Zheng, et al., Angew. Chem. Int. Ed. 2008, 47, 1938-1941.
[12] Kolb, et al., Angew. Chem. Int. Ed. 2001, 40, 2004-2021.
[13] Bumm, et al., Science 1996, 271, 1705-1707.
[14] Cui, et al., Science 2001, 294, 571-574.
[15] Xu, et al., Science 2003, 301, 1221-1223.
[16] Kushmerick, et al., J. Am. Chem. Soc. 2002, 124, 10654-10655.
[17] Reed, et al., Science 1997, 278, 252-254.
[18] Reichert, et al., Phys. Rev. Lett. 2002, 88, 176804.
[19] Pearson, et al., J. Org. Chem. 1997, 62, 1376-1387.
[20] Cai, et al., Chem. Mater. 2002, 14, 2905-2909.
[21] van Steenis, et al., Chem. Commun. 2005, 4333-4335.
[22] Castner, et al., Langmuir 1996, 12, 5083-5086.
[23] Collman, et al., Langmuir 2006, 22, 2457-2464.
[24] Tian, et al., J. Am. Chem. Soc. 2006, 128, 14748-14749.
[25] Ward, et al., Nano Lett. 2008, 8, 919-924.
[26] Braun, et al., J. Am. Chem. Soc. 2007, 129, 7760-7761.
[27] Yoo, et al., J. Colloid Interface Sci. 2007, 311, 491-496.
[28] Yeganeh, et al., J. Am. Chem. Soc. 2007, 129, 13313-13320.
[29] Landauer, IBM J. Res. Develop. 1957, 1, 223-231.
[30] Landauer, Phil. Mag. 1970, 21, 863-867.
[31] Meir, et al., Phys. Rev. Lett. 1992, 68, 2512-2515.
[32] Pearson, et al., J. Org. Chem. 1997, 62, 1376-1387.
[33] van Steenis, et al., Chem. Commun. 2005, 4333-4335.
[34] Qin, et al., Science 2005, 309, 113-115.
[35] Georgopoulos, et al., J. Toxicol. Env. Health-Pt b-Crit. Rev. 2001, 4, 341-394.
[36] Zietz, et al., Sci. Total Environ. 2003, 302, 127-144.
[37] Viguier, et al., J. Am. Chem. Soc. 2006, 128, 11370-11371.
[38] Banthia, et al., New J. Chem. 2005, 29, 1007-1010.
[39] Gattas-Asfura, et al., Chem. Commun. 2003, 2684-2685.
[40] Chan, et al., Talanta 2000, 51, 373-380.
[41] Wu, et al., Anal. Chem. 1997, 69, 2464-2470.
[42] Callahan, et al., Talanta 2002, 58, 891-898.
[43] Zheng, et al., Chem. Commun. 2002, 2350-2351.
[44] Mirkin, et al., Nature 1996, 382, 607-609.
[45] Elghanian, et al., Science 1997, 277, 1078-1081.
[46] Rosi, et al., Chem. Rev. 2005, 105, 1547-1562.
[47] Nam, et al., Science 2003, 301, 1884-1886.
[48] Han, et al., J. Am. Chem. Soc. 2006, 128, 4954-4955.
[49] Lee, et al., Angew. Chem. Int. Ed. 2007, 46, 4093-4096.
[50] Liu, et al., Chem. Mat. 2004, 16, 3231-3238.
[51] Xu, et al., Anal. Chem. 2007, 79, 6650-6654.
[52] Xu, et al., Angew. Chem. Int. Ed. 2007, 46, 3468-3470.
[53] Xue, et al., J. Am. Chem. Soc. 2008, 130, 3244-+.
[54] Seferos, et al., J. Am. Chem. Soc. 2007, 129, 15477-+.
[55] Liu, et al. Chem. Commun. 2007, 4872-4874.
[56] Zhou, et al., Angew. Chem. Int. Ed. 2008, 47, 7454-7456.
[57] Kumar, et al., J. Am. Chem. Soc. 2007, 129, 6859-6864.
[58] Gartner, et al., Science 2004, 305, 1601-1605.
[59] Jin, et al., J. Am. Chem. Soc. 2003, 125, 1643-1654.
[60] Rostovtsev, et al., Angew. Chem. Int. Ed. 2002, 41, 2596-+.
[61] Wang, et al., J. Am. Chem. Soc. 2003, 125, 3192-3193.
[62] Chan, et al., Org. Lett. 2004, 6, 2853-2855.

1. A composition comprising (a) a nanowire comprising two segments separated by a gap of about 2 nm to about 20 nm and a coating disposed along one side of the nanowire, and (b) a compound of formula (I) having a length sufficient to span the gap and having a structure I: 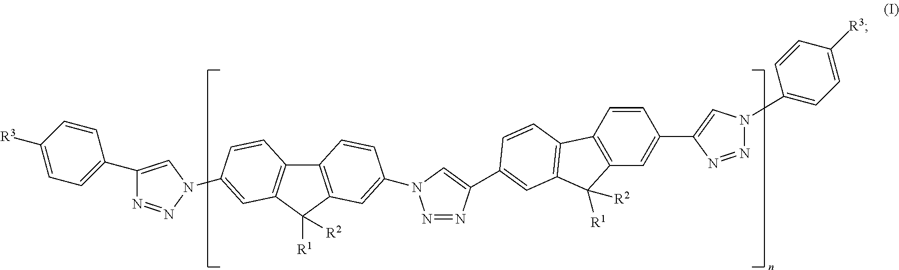
wherein R¹ and R² are independently selected from the group consisting of hydrogen, C₁-C₂₀alkyl, and C₁-C₂₀alkylaryl, each R³ is a moiety or residue of a moiety capable of forming a covalent bond or non-covalent interaction with a segment of the nanowire; and n is an integer of 1 to 20.

2. (canceled)

3. (canceled)

4. The composition of claim 1, wherein the gap is about 2 nm to about 10 nm.

5. The composition of claim 1, wherein at least one segment comprises a metal.

6. The composition of claim 5, wherein the metal comprises gold, platinum, palladium, copper, silver, nickel, titanium, or a mixture thereof.

7. (canceled)

8. The composition of claim 1, wherein at least one segment comprises a polymer.

9. (canceled)

10. (canceled)

11. The composition of claim 1, wherein the nanowire further comprises a third segment and a second gap.

12. The composition of claim 1, wherein the compound of formula (I) spans the gap of the nanowire.

13. (canceled)

14. A method of making a composition of claim 1, comprising
admixing the nanowire, a compound of formula (II), a compound of formula (III), a compound of formula (IV), and a copper (I) salt to form the compound of formula (I), wherein the compound of formula (I) spans the gap of the nanowire

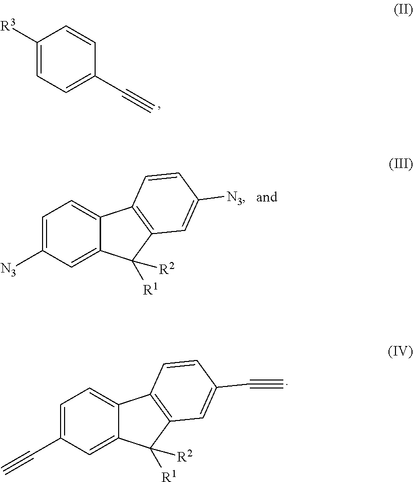

15. (canceled)

16. (canceled)

17. The method of claim 14, wherein the admixing comprises a sequence of:
(i) the compound of formula (II), the copper (I) salt, and nanowire are admixed to form a first intermediate,
(ii) the first intermediate is admixed with the compound of formula (III) in the presence of the copper (I) salt to form a second intermediate,
(iii) the second intermediate is admixed with the compound of formula (IV) in the presence of the copper (I) salt,
wherein steps (ii) and (iii) are repeated until the compound of formula (I) is formed to span the gap of the nanowire.

18. The method of claim 14, further comprising detecting the formation of the composition by monitoring an amount of current that passes through the nanowire.

19. (canceled)

20. A method of detecting copper in a sample, comprising
(a) heating the sample and a complex to determine a melting temperature of the complex in the presence of the sample, the complex comprising (i) a first oligonucleotide attached to a first nanoparticle, (ii) a second oligonucleotide attached to a second nanoparticle, and (iii) a third oligonucleotide; and
(b) comparing the melting temperature of the complex in the presence of the sample to a melting temperature of the complex in the absence of copper,
wherein when the complex has a higher melting temperature in the presence of the sample than in the absence of copper, the sample comprises copper; and
wherein each of the second and third oligonucleotide is sufficiently complementary to the first oligonucleotide to hybridize;
the second oligonucleotide is complementary to a first portion of the first oligonucleotide;
the third oligonucleotide is complementary to a second portion of the first oligonucleotide;
one of the second oligonucleotide or third oligonucleotide comprises an alkyne moiety at a terminus and the other of the second oligonucleotide or third oligonucleotide comprises an azide moiety at a terminus;
the alkyne moiety and the azide moiety react in the presence of copper to ligate the second oligonucleotide and third oligonucleotide; and
the first portion of the first oligonucleotide is sufficiently adjacent to the second portion of the first oligonucleotide to permit ligation between the first oligonucleotide and the second oligonucleotide.

21. The method of claim 20, further comprising heating the complex and sample in the presence of a copper ligand, a reducing agent, or both.

22. (canceled)

23. (canceled)

24. The method of claim 20, wherein at least one of the first nanoparticle or second nanoparticle comprises a metal.

25. (canceled)

26. The method of claim 24, wherein each of the first nanoparticle and second nanoparticle comprises gold.

27. The method of claim 20, wherein the sample comprises copper at a concentration of at least 20 μM.

28. (canceled)

29. (canceled)

30. (canceled)

31. The method of claim 20, wherein the melting temperature of the complex in the presence of the sample is at least 3° C. greater than the melting temperature of the complex in the absence of copper.

32. (canceled)

33. A method of detecting copper in a sample, comprising
(a) heating the sample and a complex comprising (1) a first oligonucleotide attached to a first nanoparticle, (2) a second oligonucleotide attached to a second nanoparticle, and (3) a third oligonucleotide; and
(b) heating the complex in the absence of copper,
wherein when the complex in the absence of copper has a change in color or absorbance before the complex in the presence of the sample has a change in color or absorbance, the sample comprises copper; and
wherein each of the second and third oligonucleotide is sufficiently complementary to the first oligonucleotide to hybridize;
the second oligonucleotide is complementary to a first portion of the first oligonucleotide;
the third oligonucleotide is complementary to a second portion of the first oligonucleotide;
one of the second oligonucleotide or third oligonucleotide comprises an alkyne moiety at a terminus and the other of the second oligonucleotide or third oligonucleotide comprises an azide moiety at a terminus;
the alkyne moiety and the azide moiety react in the presence of copper to ligate the second oligonucleotide and third oligonucleotide; and
the first portion of the first oligonucleotide is sufficiently adjacent to the second portion of the first oligonucleotide to permit ligation between the first oligonucleotide and the second oligonucleotide.

34. The method of claim 32, wherein the change in color is from colorless or light purple to red.

35. The method of claim 33, wherein the change in color is monitored by an absorbance of the complex.

36. The method of claim 33, wherein the change in absorbance indicates the copper concentration in the sample.


SEQUENCE LISTING (3 sequences)

SEQ ID NO: 1 - DNA, 40 bp, Artificial Sequence
  Synthetic nucleotide
taggaatagttataagcgtaagtcctaacgaaaaaaaaaa

SEQ ID NO: 2 - DNA, 25 bp, Artificial Sequence
  Synthetic nucleotide
ttataactattcctaaaaaaaaaaa

SEQ ID NO: 3 - DNA, 15 bp, Artificial Sequence
  Synthetic nucleotide
cgttaggacttacgc